(19) United States
(12) Patent Application Publication
(10) Pub. No.: US 2007/0172435 A1
(43) Pub. Date: Jul. 26, 2007

(54) MEDICAMENT FOR INTERNAL APPLICATION, IN PARTICULAR AGAINST CANCEROUS DISEASES

(76) Inventor: Gerd Thone, Lage (DE)

Correspondence Address: McCarter & English;Basam E Nabulsi, Financial Centre Suite 304A, 695 East Main Street, Stamford, CT, 06901-2138, US

(21) Appl. No.: 10/557,317

(22) PCT Filed: May 19, 2004
(86) PCT No.: PCT/DE04/01048

§ 371 Date: Nov. 2, 2006

(30) Foreign Application Priority Data
May 22, 2003 (DE) 103 23 758.5

(51) Int. Cl. A61K 8/00

(52) U.S. Cl. 424/59

(57) ABSTRACT

The invention relates to a medicament for internal application, in particular against cancerous diseases. The medicament comprises polydimethylsiloxane and surfactants, whereby the proportion of polydimethylsiloxane is greater than the proportion of surfactants.

The present invention relates to a drug for internal use, more specifically for treating cancerous conditions.

There has long been a need for a drug against cancerous conditions. Although drugs are known and utilized, they often have serious side effects. Also, the currently utilized drugs are very expensive, not least because they are complex to manufacture. Therefore, there is a continuous need for well-tolerated low-cost drugs in this field.

This is where the invention sets in. It is its object to indicate a drug for internal use, more specifically for treating cancerous conditions that permits to most effectively attack cancer tumors. The drug should have least side effects and be low in cost.

This object is solved by a drug comprising polydimethylsiloxane and surfactants with the fraction of polydimethylsiloxane exceeding the fraction of surfactants.

The peculiarity of the invention is that the effect of the invention is only achieved by adding surfactants. For, by mixing the polydimethylsiloxane with surfactants, the body is given the possibility to emulsify the siloxane and to make it usable. This is achieved by the surfactants that cause the molecule structure of the polydimethylsiloxane to break up. Related to the weight of the solution, the fraction of surfactants can be significantly smaller than the fraction of polydimethylsiloxane.

Anionic, amphoteric and non-ionic surfactants can be used, with the mass fraction of the anionic surfactants exceeding the mass fraction of the amphoteric and non-ionic ones.

In this context, it should be noted that most naturally occurring silicon, even rock crystal, which consists of pure silicon, have no bioactive effect. As already mentioned, this property is only achieved by adding surfactants.

Polydimethylsiloxane is made from raw silicon. Raw silicon is obtained from sand and coal and is further processed to the desired silicones in a continuous process. Natural gas or petroleum serves to produce methanol (synthesis gas), another starting material for the synthesis of silicones. Chlorine, which is supplied to the process in the form of HCl, is obtained by the electrolysis of rock salt solutions.

In the first step, methanol is converted together with HCl to chloromethane (synthesis of chloromethane). Next, a mixture of raw silanes is obtained by reacting chloromethane with silicon (synthesis of chlorosilane). These raw silanes are separated by distillation, with dichlorodimethylsilane (CH3)2SiCl2 being converted by hydrolysis to polydimethylsiloxane.

In principle, it is possible to inhale the drug of the invention. For inhalation, the drug is nebulized in an apparatus using appropriate nebulizing techniques. Diverse apparatus are available for this purpose:
1. Pneumatic Venturi nozzle nebulizer:
   a) direct nebulization
   b) with an aerosol reservoir
   c) with an overpressurized solution to be inhaled.
2. Mechanical one-substance nozzle nebulizer
3. Ultrasonic nebulizing
4. Ultrasonic pressure through perforated screen
5. Ultrasound-operated perforated membrane
6. Electromechanical pressure through perforated membrane

Inhalation through an inhalation mask as well as with a mouthpiece or a nosepiece has been found to be well suited for this purpose.

In addition to the constituent substances mentioned, aluminium silicate, more specifically natural zeolite with a grain size in the range of 10 μm to 70 μm, more specifically of 40 μm, and dolomite powder with a grain size in the range of 2 μm to 30 μm, more specifically of 10 μm, may be added. A powder is thus obtained that may be administered in capsules.

The framework of the zeolite crystal lattice is mainly built from SiO₄ tetrahedrons. It comprises empty spaces containing ions such as sodium, potassium and calcium that can be readily interchanged and exchanged with their substrate environment. In living organisms, this mineral-specific crystal structure (cage structure) of zeolite has the excellent property of binding (absorbing) toxins such as ammonia and other nitrogen compounds but also heavy metals and to eliminate them through the digestive tract. The eliminated toxins are replaced by minerals the body urgently needs. Thus, the organism's homeostasis, more specifically the mineral metabolism, is maintained or reestablished.

Accordingly, delicate organ systems such as the brain, the nervous system, the hormonal system, the immune system, the liver, the kidneys and so on are not only protected against toxic damage, their resistance to toxic pathogenic influences is also increased.

Like silicon, zeolite moreover has a positive stimulating influence upon the entire metabolism and on the growth and healing processes of the organism.

Thanks to its open molecule structure, zeolite is further capable of absorbing large amounts of liquid. This presents an advantage as it permits to form a flowable powder in spite of its being mixed with the above mentioned additional constituent substances.

In the final product, the additional constituent substances may for example comprise the following fractions:
aluminium silicate: 50 to 90%, more specifically 70% by weight,
dolomite powder: 5 to 45, more specifically 25% by weight.

The thus formed mixture is a flowable powder and can be processed into a drug in a variety of manners. It is for example possible to encapsulate the powder mixture, for example in the form of the widely used gelatine capsules that dissolve in the digestive tract. It is moreover readily possible to compress the powder mixture into tablets that may be taken with or without liquid.

It has been found that, although the solutions are effective against cancer cells, they do not affect healthy cells.

Clinical testing showed that the drug of the invention has the following effects:
improved tolerance to chemotherapy and radiotherapy,
tumor growth suppression,
tumor induration (mineralization),
partial tumor encapsulation and reduction,
improved general condition,
elimination of inflammatory processes as a side effect of radiotherapy and chemotherapy, e.g., of the mucous membranes of the mouth.

Further, first tests showed that the drug of the invention is virostatic. First successful outcomes in the treatment of AIDS (HIV) and SARS have been achieved. In this respect, the applicant reserves the right to file a continuation-in-part application.

1. A drug for internal use, more specifically against cancerous conditions, characterized by the constituent substances polydimethylsiloxane and surfactants, with the fraction of polydimethylsiloxane exceeding the fraction of surfactants.

2. The drug as set forth in claim 1, characterized in that the mixture of the two constituent substances contains a fraction of polydimethylsiloxane in the range of about 90-99.9, more specifically of 99% by weight, and a fraction of surfactants in the range of 0.1-10, more specifically of 1% by weight.

3. The drug as set forth in claim 1 or claim 2, characterized in that aluminium silicate, more specifically natural zeolite with a grain size in the range of 10 μm to 70 μm, more specifically of 40 μm, and dolomite powder with a grain size in the range of 2 μm to 30 μm, more specifically of 10 μm, are added.

4. The drug as set forth in claim 3, characterized in that the additional constituent substances comprise the following fractions in the final product:
aluminium silicate: 50 to 90%, more specifically 70% by weight,
dolomite powder: 5 to 45, more specifically 25% by weight.

5. The drug as set forth in any one of the claims 1 through 4, characterized in that it contains anionic, amphoteric and non-ionic surfactants, with the mass fraction of the anionic surfactants exceeding the mass fraction of the amphoteric and non-ionic ones.

6. Use of polydimethylsiloxane and of surfactants for manufacturing a drug for internal use, more specifically against cancerous conditions.

7. Use of polydimethylsiloxane and of surfactants for manufacturing a drug as set forth in claim 6, characterized in that the mixture of the two constituent substances contains a fraction of polydimethylsiloxane in the range of about 90-99.9, more specifically of 99% by weight, and a fraction of surfactants in the range of 0.1-10, more specifically of 1% by weight.

8. Use of polydimethylsiloxane and of surfactants for manufacturing a drug as set forth in claim 6 or claim 7, characterized in that aluminium silicate, more specifically natural zeolite with a grain size in the range of 10 μm to 70 μm, more specifically of 40 μm, and dolomite powder with a grain size in the range of 2 μm to 30 μm, more specifically of 10 μm, are added.

9. Use of polydimethylsiloxane and of surfactants for manufacturing a drug as set forth in any one of the claims 6 through 8, characterized in that the additional constituent substances comprise the following fractions in the final product:
aluminium silicate: 50 to 90%, more specifically 70% by weight,
dolomite powder: 5 to 45, more specifically 25% by weight.

10. Use of polydimethylsiloxane and of surfactants for manufacturing a drug as set forth in any one of the claims 6 through 9, characterized in that it contains anionic, amphoteric and non-ionic surfactants, with the mass fraction of the anionic surfactants exceeding the mass fraction of the amphoteric and non-ionic ones.
